(19) United States
(12) Patent Application Publication
(10) Pub. No.: US 2025/0121196 A1
(43) Pub. Date: Apr. 17, 2025

(54) EVOKED RESPONSE-GUIDED MULTISITE DEEP BRAIN STIMULATION

(71) Applicant: Boston Scientific Neuromodulation Corporation, Valencia, CA (US)

(72) Inventor: G. Karl Steinke, Valencia, CA (US)

(21) Appl. No.: 18/896,427

(22) Filed: Sep. 25, 2024

Related U.S. Application Data
(60) Provisional application No. 63/590,277, filed on Oct. 13, 2023.

(51) Int. Cl. A61N 1/36

(52) U.S. Cl. CPC A61N 1/36139; A61N 1/36067

(57) ABSTRACT

Systems and methods for providing evoked response-guided multisite deep brain stimulation (DSB) at various brain targets are disclosed. An exemplary system comprises at least one multielectrode lead, an electrostimulator to provide electrostimulation to a neural target, a sensing circuit to sense an evoked response (ER) to electrostimulation, and a controller circuit. In response to electrostimulation of a first neural target according to a first stimulation setting, the controller circuit collects an ER from a first sensing location, and determines or adjusts a second stimulation setting for stimulating a second neural target based on the sensed ER at the first neural target. The second neural target can be stimulated according to the second stimulation setting to modulate the ER to the electrostimulation at the first neural target, and to produce a desired therapeutic outcome in the patient.

### CLAIM OF PRIORITY

This application claims the benefit of U.S. Provisional Application No. 63/590,277, filed on Oct. 13, 2023, which is hereby incorporated by reference in its entirety.

### TECHNICAL FIELD

This document relates generally to medical systems, and more particularly, but not by way of limitation, to multisite deep brain stimulation (DBS) systems and methods based on evoked responses.

### BACKGROUND

Medical devices may include therapy-delivery devices capable of delivering a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

It has been proposed to use evoked potentials (also referred to as evoked responses or ERs) to guide neurostimulation therapy. For example, Evoked Resonant Neural Activity (ERNA) has been proposed as a feedback signal for controlling DBS delivery. ERNA may also be referred to by other names such as DBS Local Evoked Potentials (DLEP), Evoked oscillatory neural responses (EONR), and other terms. Evoked potentials, including ERNA, may be present in other indications and anatomical structures or locations.

DBS may improve motor symptoms in some patients with advanced Parkinson's Disease (PD), and other motor and non-motor disorders. Stimulation leads/electrodes for PD treatment are commonly implanted in subthalamic nucleus (STN) or globus pallidus internus (GPi), although other neural targets, such as pedunculopontine nucleus (PPN) and posterior subthalamic area (PSA), and others, have been shown as effective targets for Parkinsonian tremor control and control of other Parkinson's symptoms in patients. However, optimal target(s) of DBS to manage PD and other movement and non-movement disorders may vary across patients, and there is no best “universal” DBS target. A DBS system capable of providing versatile and flexible stimulation at multiple neural targets is desired to accommodate the needs of patients with different conditions.

### SUMMARY

Disclosed herein are systems and methods of multisite neuromodulation, in particular DBS of multiple brain targets, such as STN and GPi. Multisite DBS may recruit neural pathways associated with multiple neural targets, effectuate a broader stimulation field than a single local target, and maximize therapeutic effect and/or minimize side effects. In accordance with various embodiments, different neural targets may be stimulated substantially simultaneously (within a specific time delay), sequentially with a time offset, or in accordance with a predetermined stimulation pattern. In response to electrostimulation of a neural target, an evoked response (ER) may be sensed locally, at a location distant away from the stimulation site, or both, and used to feedback control the DBS, or to determine an optimal DBS setting. In an embodiment, an ER to stimulation at a first neural target (hereinafter “ER₁”) may be used to determine or adjust a stimulation setting at a different second neural target. Electrostimulation at the second neural target may modulate ER₁, and produce a “compound” ER, which can be analyzed to determine if a desired therapeutic outcome has been achieved, such as a high therapeutic effect and low side effect. The ER modulation may be that, in one example, the electrostimulation of the second target boosts ER₁, thus producing an enhanced compound ER. In another example, the electrostimulation of the second target reduces ER₁, thus producing a diminished compound ER. The multisite DBS as described herein may conveniently and more effectively establish a desirable compound ER, which may lead to better therapeutic outcome of the patient.

An example (e.g., “Example 1”) of a neuromodulation system may include: at least one lead including a plurality of electrodes; an electrostimulator configured to provide electrostimulation to neural targets of a patient via one or more of the plurality of electrodes of the at least one lead; a sensing circuit configured to sense an evoked response (ER) to the electrostimulation; and a controller circuit operably connected to the electrostimulator and the sensing circuit, the controller circuit configured to: in response to the electrostimulation delivered to a first neural target in accordance with a first stimulation setting, collect an ER sensed via the sensing circuit from a first sensing electrode positioned at a first sensing location; based at least in part on the sensed ER to the electrostimulation at the first neural target, determine or adjust a second stimulation setting for stimulating a second neural target of the patient different from the first neural target; generate a control signal to the electrostimulator to provide electrostimulation at the second neural target in accordance with the second stimulation setting to modulate the sensed ER to the electrostimulation at the first neural target, and to produce a desired therapeutic outcome in the patient; and collect an ER to the electrostimulation at the second neural target sensed via the sensing circuit from a second sensing electrode positioned at a second sensing location.

In Example 2, the subject matter of Example 1 optionally includes the controller circuit that may be configured to control the electrostimulator to deliver respective electrostimulations to the first and the second neural targets substantially simultaneously in accordance with respective the first and the second stimulating settings.

In Example 3, the subject matter of any one or more of Examples 1-2 optionally includes the controller circuit that may be configured to control the electrostimulator to deliver respective electrostimulations to the first and the second neural targets sequentially with a specific time offset in accordance with respective the first and the second stimulating settings.

In Example 4, the subject matter of any one or more of Examples 1-3 optionally includes the first and the second neural targets of electrostimulation which are distinct brain targets in a specific hemisphere of the brain.

In Example 5, the subject matter of Example 4 optionally includes the first neural target of electrostimulation being a subthalamic nucleus (STN) target, and the second neural target of electrostimulation being a globus pallidus internus (GPi) target.

In Example 6, the subject matter of Example 5 optionally includes the first sensing electrode that may be positioned at a GPi sensing location to sense the ER to electrostimulation of the STN target, and the second sensing electrode that may be positioned at an STN sensing location to sense the ER to electrostimulation of the GPi target.

In Example 7, the subject matter of any one or more of Examples 1-6 optionally includes an external electrostimulator operatively coupled to one or more cortical electrodes or one or more stereotactic electrodes to provide cortical stimulation to modulate one or more of the sensed ER to the electrostimulation at the first neural target or the sensed ER to the electrostimulation at the second neural target, and to produce the desired therapeutic outcome in the patient.

In Example 8, the subject matter of any one or more of Examples 1-7 optionally includes the electrostimulator that may be electrically coupled to a multielectrode lead, the electrostimulator configured to provide electrostimulation to the first neural target via a first electrode on the multielectrode lead, and to provide electrostimulation to the second neural target via a second electrode on the same multielectrode lead.

In Example 9, the subject matter of any one or more of Examples 1-8 optionally includes the at least one lead that may include distinct first and second leads each including one or more electrodes, wherein the electrostimulator is configured to provide electrostimulation to the first neural target via a first electrode on the first lead, and to provide electrostimulation to the second neural target via a second electrode on the second lead.

In Example 10, the subject matter of any one or more of Examples 1-9 optionally includes at least one of the first or the second sensing electrode that may be selected from the plurality of electrodes of the at least one lead.

In Example 11, the subject matter of any one or more of Examples 1-10 optionally includes at least one of the sensed ER to the electrostimulation at the first neural target or the sensed ER to the electrostimulation at the second neural target that may include an electrocorticography (ECoG) or a stereoelectroencephalography (sEEG) sensed via one or more cortical electrodes or one or more stereotactic electrodes.

In Example 12, the subject matter of any one or more of Examples 1-11 optionally includes the controller circuit that may be configured to determine or adjust the second stimulation setting such that the electrostimulation at the second neural target in accordance with the second stimulation setting reduces the ER to the electrostimulation at the first neural target.

In Example 13, the subject matter of any one or more of Examples 1-12 optionally includes the controller circuit that may be configured to determine or adjust the second stimulation setting such that the electrostimulation at the second neural target in accordance with the second stimulation setting enhances the ER to the electrostimulation at the first neural target.

In Example 14, the subject matter of any one or more of Examples 1-13 optionally includes the controller circuit that, to determine or adjust the second stimulation setting based at least in part on the sensed ER to the electrostimulation at the first neural target, is configured to: evaluate a therapeutic outcome using the sensed ER to the electrostimulation at the first neural target; and based on the evaluation of the therapeutic outcome, determine or adjust the second stimulation setting including one or more stimulation parameters or one or more electrodes selected from the plurality of electrodes of the at least one lead for electrostimulation.

In Example 15, the subject matter of Example 14 optionally includes the controller circuit is configured to evaluate the therapeutic outcome including to compare a spatial distribution of the sensed ER to the electrostimulation at the first neural target to a desired ER spatial distribution, and to determine or adjust the second stimulation setting to reduce a discrepancy between the spatial distribution of the sensed ER to the electrostimulation at the second neural target and the desired ER spatial distribution.

Example 16 is a method of providing neurostimulation to neural targets of a patient via a neuromodulation system that comprises an electrostimulator and at least one lead coupled thereto. The method comprises steps of: delivering electrostimulation to a first neural target in accordance with a first stimulation setting using the electrostimulator; sensing an evoked responses (ER) to the electrostimulation at the first neural target via a first sensing electrode positioned at a first sensing location; via a controller circuit, determining or adjusting a second stimulation setting for stimulating a second neural target of the patient different from the first neural target based at least in part on the sensed ER to the electrostimulation at the first neural target; delivering electrostimulation to the second neural target in accordance with the second stimulation setting to modulate the sensed ER to the electrostimulation at the first neural target, and to produce a desired therapeutic outcome in the patient; and sensing an ER to the electrostimulation at the second neural target via a second sensing electrode positioned at a second sensing location.

In Example 17, the subject matter of Example 16 optionally includes the electrostimulation of the first neural target and the electrostimulation of the second neural target that may be delivered substantially simultaneously, or sequentially with a specific time offset, in accordance with respective the first and the second stimulating settings.

In Example 18, the subject matter of any one or more of Examples 16-17 optionally includes the first neural target of electrostimulation being a subthalamic nucleus (STN) target in a hemisphere of a brain, and the second neural target of electrostimulation being a globus pallidus internus (GPi) target in the hemisphere of the brain.

In Example 19, the subject matter of any one or more of Examples 16-18 optionally includes providing cortical stimulation via an external electrostimulator operatively coupled to one or more cortical electrodes or one or more stereotactic electrodes, the cortical stimulation modulating one or more of the sensed ER to the electrostimulation at the first neural target or the sensed ER to the electrostimulation at the second neural target, and producing the desired therapeutic outcome in the patient.

In Example 20, the subject matter of any one or more of Examples 16-19 optionally includes the at least one lead that may include distinct first and second leads each including one or more electrodes, wherein the electrostimulation to the first neural target is delivered via a first electrode on the first lead, wherein the electrostimulation to the second neural target is delivered via a second electrode on the second lead.

In Example 21, the subject matter of any one or more of Examples 16-20 optionally includes at least one of the sensed ER to the electrostimulation at the first neural target or the sensed ER to the electrostimulation at the second neural target that may include an electrocorticography (ECoG) or a stereoelectroencephalography (sEEG) sensed via one or more cortical electrodes or one or more stereotactic electrodes.

In Example 22, the subject matter of any one or more of Examples 16-21 optionally includes the second stimulation setting that may be determined or adjusted such that the electrostimulation at the second neural target in accordance with the second stimulation setting produces a desired modulation of the ER to the electrostimulation at the first neural target, the desired modulation including reducing the ER to the electrostimulation at the first neural target, or enhancing the ER to the electrostimulation at the first neural target.

In Example 23, the subject matter of any one or more of Examples 16-22 optionally includes comparing a spatial distribution of the sensed ER to the electrostimulation at the first neural target to a desired ER spatial distribution, wherein the second stimulation setting is determined or adjusted to reduce a discrepancy between the spatial distribution of the sensed ER to the electrostimulation at the second neural target and the desired ER spatial distribution.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates a parasagittal slice through the nonhuman primate brain showing major anatomical pathways involved in STN DBS.

FIG. 8 illustrates, by way of example and not limitation, a neuromodulation system that can provide ER-guided multisite DBS at various brain targets.

FIGS. 9A-9B illustrate, by way of example and not limitation, a single-lead configuration and a dual-lead configuration for providing multisite DBS and sensing respective ERs.

FIG. 10 illustrates, by way of example and not limitation, a display of a comparision between a user-defined ER target location and a calculated ER distribution center.

FIGS. 11A-11D illustrate, by way of example and not limitation, stimulation patterns across electrodes or groups of electrodes for use in mutlisite DBS.

FIG. 12 illustrates, by way of example and not limitation, a method of providing ER-guided multisite DBS at various brain targets.

FIG. 13 illustrates generally a block diagram of an example machine upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

In device-based neuromodulation therapy, finding an “optimal” stimulation target is an important yet challenging technical problem. It is generally recognized that there is no universal optimal target or desired evoked response due to differences in patient condition or disorder, anatomy (anatomical target), lead and trajectory, center (imaging equipment position, etc.), surgeon (how they implant and position lead, their preferences for lead placement), or symptoms to improve (e.g., tremor to cognitive skill improvement). In DBS for Parkinson's Disease (PD) management, there is no consensus on the “best” DBS target across PD patients. While DBS effectively treats several motor symptoms of PD, limitations remain in side effect profile, management of non-motor symptom, accurate lead placement with intraoperative testing, and selection of optimal stimulation parameters. For example, although STN and GPi are common targets of DBS for treating PD and both GPi and STN DBS improve patient motor function and activities of daily living, some PD patients may benefit more from STN DBS than GPi DBS, while some others may benefit more from GPi DBS than STN DBS. STN DBS generally allows greater reduction in medication for patients, but may cause motor side effects such as speech impairment and dyskinesia in some patients. GPi DBS generally provides greater relief from psychiatric symptoms, but may also causer certain side effects in some other patients. The present inventor has recognized an unmet need for neuromodulation systems capable of providing versatile DBS to multiple targets in a controlled, selective manner to meet specific needs of individual patients.

Evoked responses (ERs) have been used to guide implantation procedure (e.g., lead placement or electrode positioning), or programming of device settings (e.g., sensing parameters or neurostimulation parameters). ERs are generated by neural activities in response to applied stimuli and reveal information about neural connectivity and function. ERs may be caused by diagnostic stimulation or therapeutic stimulation, or both. Stimulation may be located where placing evoking pulses gets a desired response such as to maximize ERNA, where listening for responses gets a desired response (e.g., maximize ERNA, where placing lead is desired (e.g., best for therapy, or where placing stimulation on the lead is desired (e.g., maximize therapy and/or minimize/counter side effects).

In DBS for PD management, DBS generates EPs in both local (subcortical) and remote (cortical) regions of the nervous system based on neuronal and axonal stimulation. ERs have shown potential to improve programming, reveal relevant mechanisms, and identify circuits involved in DBS for PD. ERs may be sensed from an anatomical target, such as like a volume (e.g., STN), a collection of fibers, a sub-region (motor STN, or dorsolateral STN), a volume of interest described within or related to a particular patient's brain such as from atlas or aggregate prior information, or a “point” that may be described by optimizing a stimulation location. The ERs may be used to optimize stimulation settings in multisite DBS, including, for example, lead placement, electrode selection, and programming of sensing parameters or neurostimulation parameters.

Various embodiments described in this document implement multisite neuromodulation, particularly multisite DBS of various brain targets including, for example, STN and GPi. The multisite DBS as described herein may recruit neural pathways associated with multiple neural targets being stimulated, effectuate a broader stimulation field rather than a single local target, and maximize therapeutic effect and/or minimize side effects. In an example, GPi DBS may strengthen the stimulation field established by STN DBS, thereby producing a more favorable therapeutic outcome. In accordance with some examples, multisite DBS may facilitate establishing a target or desirable ER by modulating the properties and stimulation timing in multiple targets. For example, when STN DBS and GPi DBS are programmed to produce the same effect, STN DBS may build a “baseline” ER, while GPi DBS may be programmed to boost or maximize the baseline ER. Alternatively, STN DBS and GPi DBS may be programmed to produce opposite effects, such that GPi DBS may be programmed to reduce or minimize the baseline ER established by STN DBS. The desired ERs can be mapped to optimized therapeutic stimulation settings.

The multisite DBS as described herein enables more versatile therapy control. The multisite DBS (e.g., STN DBs and GPi DBS) may be delivered substantially simultaneously, sequentially with a time offset, or in accordance with a predetermined stimulation pattern. The multisite DBS may be implemented using multiple distinct volumes (i.e., targets) of stimulation, or multipolar stimulation. In certain examples, one or more leads may be placed in the brain to deliver multisite DBS. Stimulation settings, such as stimulation target selection, time offset between stimulations at different targets, and stimulation parameter values, may be titrated for individual patient. ERs to stimulations at respective neural targets may be sensed at locations distant away from the stimulation target. In an example, ER to STN DBS may be sensed at a location at or proximal to GPi in a brain hemisphere, and ERs to GPi DBS may be sensed at a location at or proximal to STN in the same brain hemisphere. The ERs may be used to feedback control the DBS, and to optimize personalized multisite DBS, including lead placement and stimulation parameter setting.

The multisite DBS as described herein may also help compare and contrast various target candidates for new indications in DBS by exploring functional or structural connectivity patterns. For example, when DBS is used for managing patients with treatment-resistant depression (TRD), one challenge is to determine between subcallosal cingulate cortex (SCC) and ventral capsule/ventral striatum (VC/VS) a better target of DBS. Multisite DBS implementation as described in the present document may provide a test platform for evaluating and contrasting treatment outcomes associated with respective neural targets.

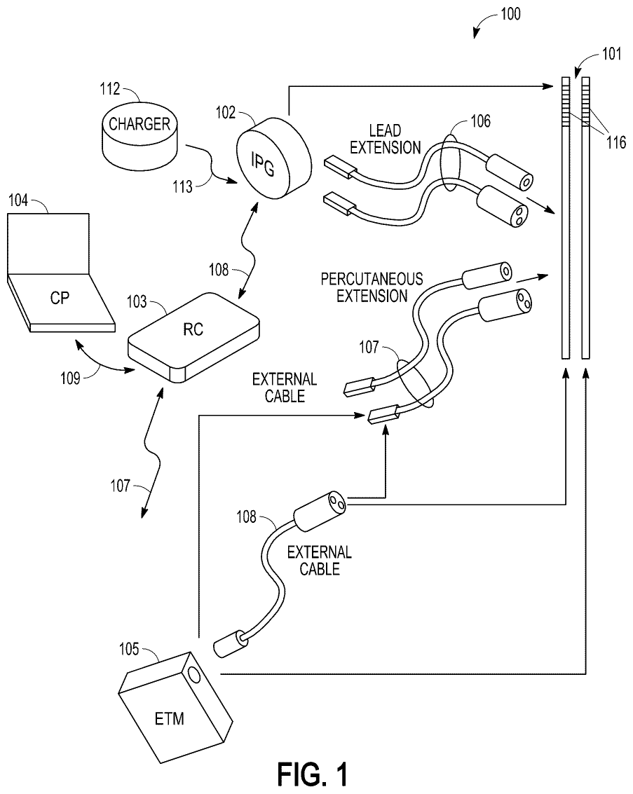

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

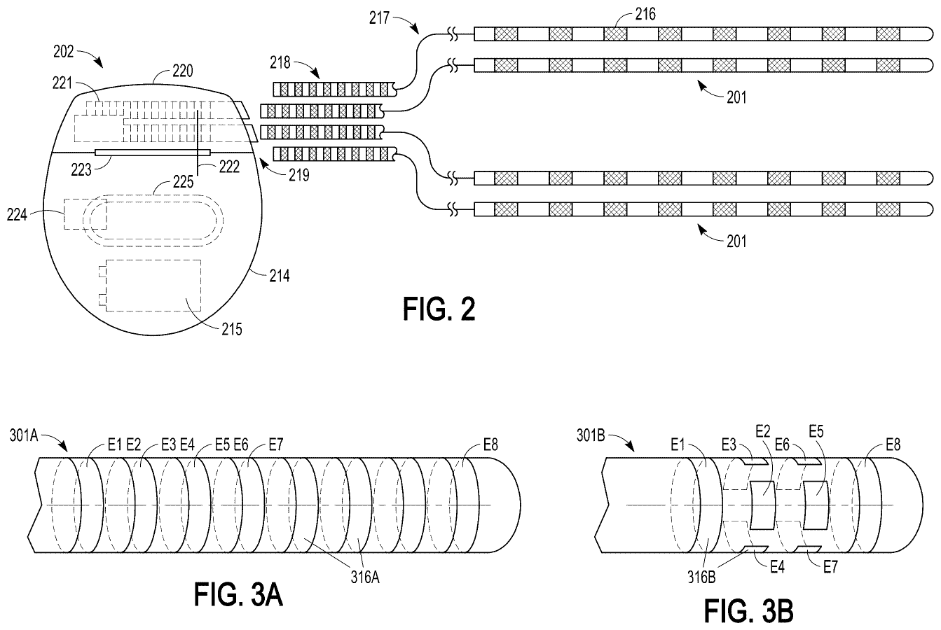

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more rows of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

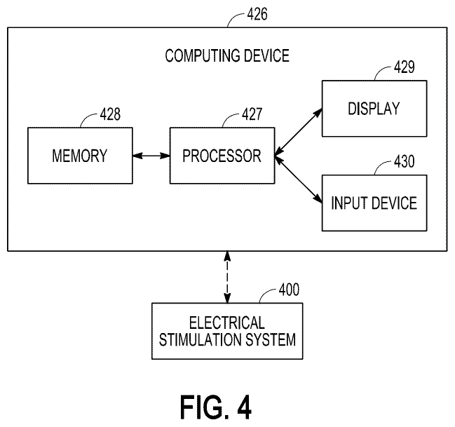

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

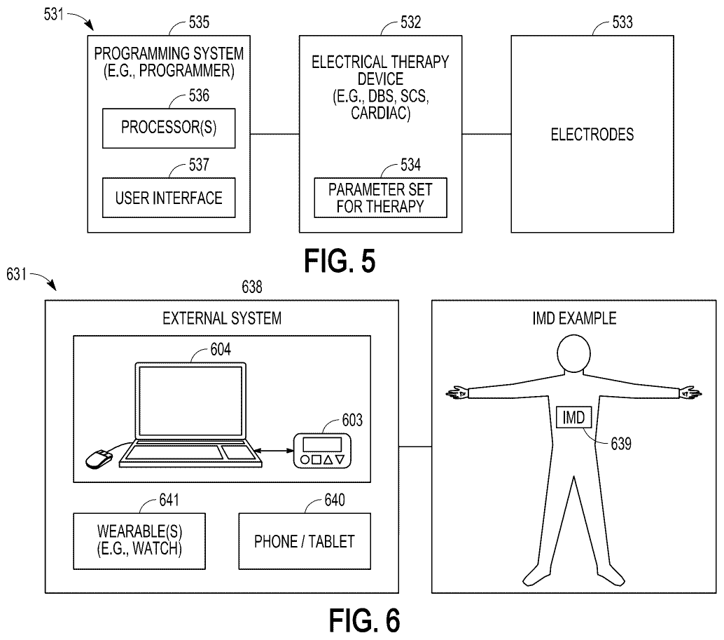

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

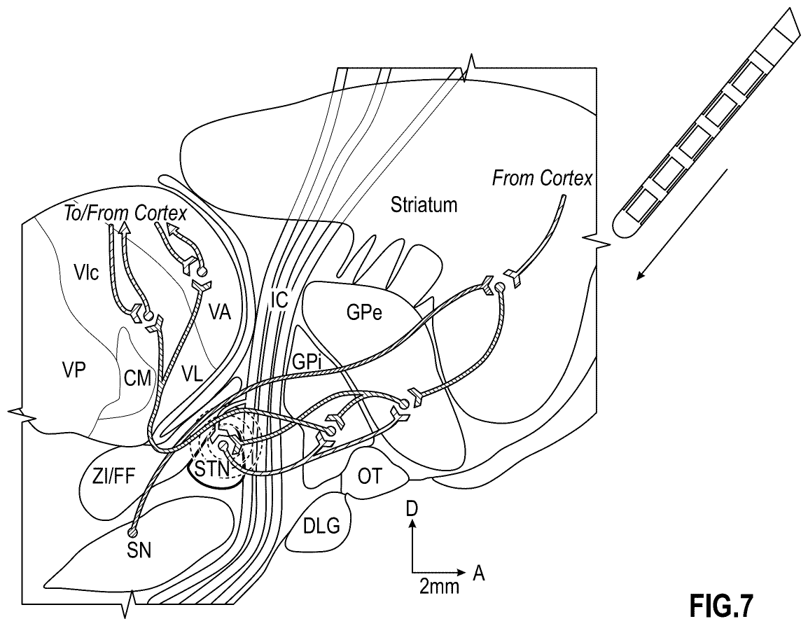

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

This document describes DBS of multiple brain targets, such as STN and GPi, for PD management. Anatomy of neural circuit elements by which the basal ganglia are connected to regions of the thalamus and cortex is important for an understanding of the mechanisms that are involved in ER generation responsive to DBS at neural targets such as STN and GPi. FIG. 7 illustrates a parasagittal slice through the nonhuman primate brain. The figure, reproduced from Devergnas and Wichmann Frontiers in systems neuroscience (2011) 5, 30, shows the major anatomical pathways involved in subthalamic nucleus (STN) DBS, including the internal capsule (IC), globus pallidus internus (GPi), and globus pallidus externus (GPe). Excitatory glutamatergic connections are shown as red lines, inhibitory GABAergic connections are shown as black lines, and dopaminergic connections are shown as green lines. The blue concentric lines symbolize the spread of the STN DBS. As shown in FIG. 7, STN is located in a very crowded region of the brain. The STN is part of the “indirect” pathway of the basal ganglia. This pathway links the principal input structure of the basal ganglia, the striatum, to the output structures, the GPi, and the substantia nigra pars reticulata (SNr), via the external segment of the GPe and the STN. During DBS procedures, at least one lead can be implanted into the brain such that certain electrodes are proximal to brain regions like the STN. Typically, STN DBS electrodes are implanted into the central STN. The most ventral electrode tends to be implanted at the ventral border of the STN, or may extend into the dorsal SNr. Depending on the contact separation of the specific electrode used, the top contact is either located in the zona incerta (ZI) or in the central thalamus. Subsequently, continuous high-frequency stimulation is delivered via an IPG which can be externally programmed.

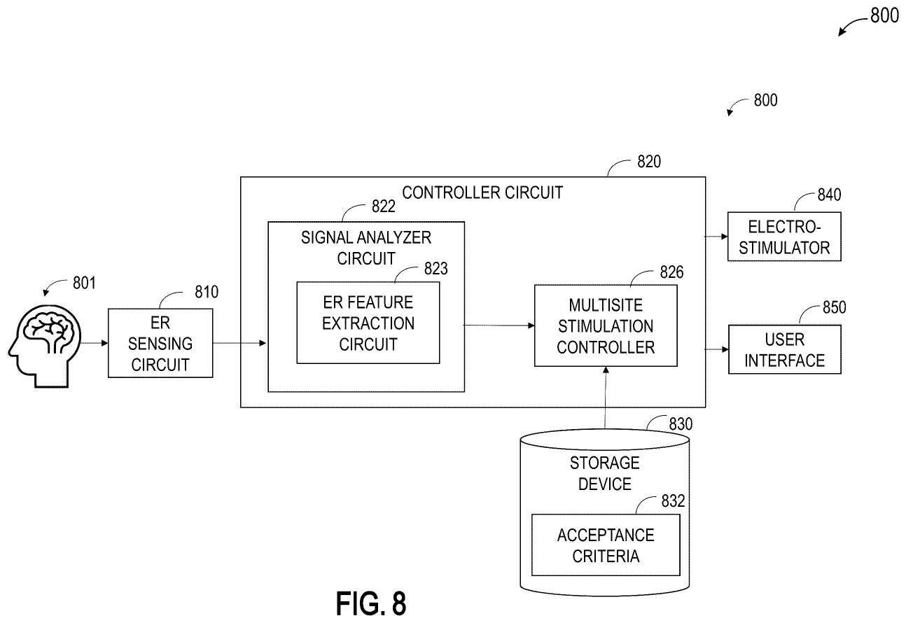

FIG. 8 illustrates an example of a neuromodulation system 800 configured to provide ER-guided multisite DBS at various brain targets. The system 800 includes a sensing circuit 810, a controller circuit 820, a storage device 830, an electrostimulator 840, and a user interface 850. Portions of the system 800 may be implemented in the IPG 102 or the CP 104.

The sensing circuit 810 may sense ERs to DBS delivered to a neural target in a brain of a patient 801, such as STN or GPi in a brain hemisphere. The ER may be sensed from a sensing location of the brain via one or more sensing electrodes or sensors. The sensing circuit 810 may be electrically coupled to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 840, which may provide multisite DBS at respective different neural targets. The ring electrodes and/or the segmented electrodes may be configured as sensing electrodes for sensing ERs, or as stimulating electrodes for delivering stimulation pulses. In various examples, the ERs may be sensed in accordance with a stimulating-sensing electrode configuration representing a correspondence between stimulation electrodes and the sensing electrodes used for sensing ER on the same lead.

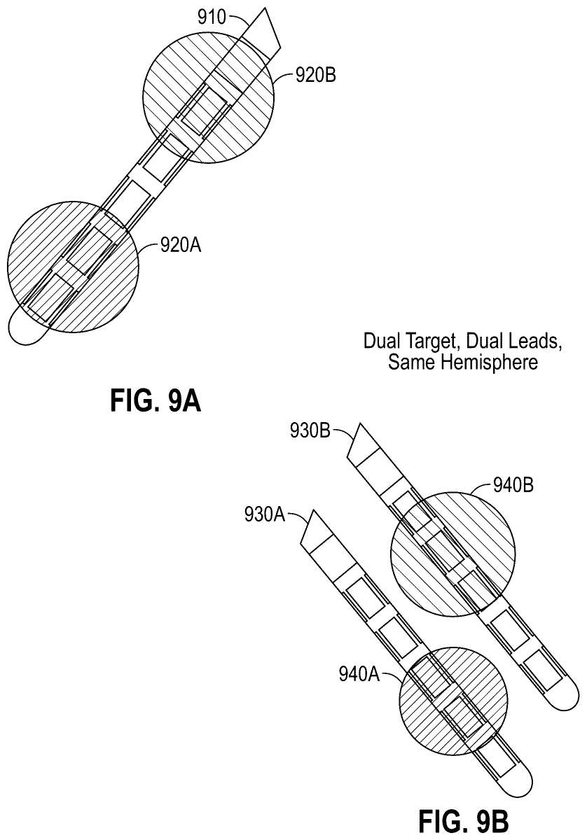

The stimulating electrodes for delivering DBS and the sensing electrodes for sensing ER may be selected from electrodes associated with the same lead, or alternatively, from electrodes associated with different leads. Referring to FIGS. 9A-9B, by way of example and not limitation, a single-lead configuration and a dual-lead configuration are illustrated to provide multisite DBS and sensing respective ERs. FIG. 9A illustrates an example of a single lead 910 that may be implanted in a hemisphere of a brain during a DBS procedure. The lead 910 may be positioned such that a first electrode or set of electrodes are at or proximal to a first stimulation target 920A (e.g., STN), and a second electrode or set of electrodes are at or proximal to a second stimulation target 920B (e.g., GPi) at the same hemisphere. DBS of the first stimulation target (e.g., STN) may be delivered in accordance with a first stimulation setting. DBS of the second stimulation target (e.g., GPi) may be delivered in accordance with a second stimulation setting. The first and second stimulation settings may each include values of a plurality of stimulation parameters such as pulse amplitude, pulse width, pulse rate or frequency, etc. The first and the second stimulation settings may also include respective timings to initiate stimulation. In accordance with various embodiments, DBS of the first and the second neural targets may be delivered substantially simultaneously, sequentially with a time offset, or in accordance with a predetermined stimulation pattern. DBS of the first stimulation target 920A may establish a first stimulation field in an area around the first stimulation target. DBS of the second stimulation target 920B may establish a second stimulation field in an area around the second stimulation target. As will be discussed further below, the second stimulation setting may be determined based on ER to DBS at the first stimulation (ER₁), and the DBS of the second neural target may modulate ER₁, such as to boost the ER₁ to produce an enhanced compound ER, or alternatively to reduce the ER₁ to produce a diminished compound ER.

Evoked responses to DBS at a particular neural target may be sensed using one or more electrodes on the lead 910 at a location distant away from the DBS stimulation target. In an example, an ER to STN DBS may be sensed at a location at or proximal to GPi. In another example, an ER to GPi DBS may be sensed at a location at or proximal to STN in the same brain hemisphere. FIG. 9B illustrates an example of dual leads 930A and 930B both implanted in the same hemisphere of a brain. The lead 930A is such positioned that a first electrode or set of electrodes are at or proximal to a first stimulation target 940A (e.g., STN). The lead 930B is such positioned that a second electrode set of electrodes are at or proximal to a second stimulation target 940B (e.g., GPi) at the same hemisphere. Evoked responses to DBS at a particular neural target may be sensed using one or more electrodes on either the lead 930A or the lead 930B, at a location distant away from the DBS stimulation target. In an example, the lead 930A may deliver STN DBS in accordance with a first stimulation setting, which may establish a first stimulation field in an area around the first stimulation target 940A, where ER to STN DBS may be sensed at a location at or proximal to GPi via electrodes on either lead 930A or 930B. The lead 930B may deliver GPi DBS in accordance with a second stimulation setting, which may establish a second stimulation field in an area around the second stimulation target 940B, where ER to GPi DBS may be sensed at a location at or proximal to STN via electrodes on either lead 930A or 930B.

In some examples, at least some sensing electrodes or the stimulating electrodes may be selected from electrodes other than electrodes on an implantable lead (such as the leads 910, 930A, or 930B), such as skin patch electrodes. In some examples, intracranial brain activity, such as intracranial electroencephalograph (iEEG) may be recorded via electrode placed directly on the neocortex to record electrocorticography (ECoG), or placed intracortically to record stereoelectroencephalography (sEEG). Other nomenclatures and methods of describing the evoking and recording electrodes may be used, including those which involve multiple evoking electrodes with proportioned or fractionalized current, or Multiple Independent Current Control (MICC) to generate precise control to refine the size and shape of the stimulation field, designed to customize therapy for individual patients.

In some examples, DBS may be provided using an external electrostimulator. The external electrostimulator may be operatively coupled to one or more cortical electrodes or one or more stereotactic electrodes to provide cortical stimulation. In some examples, electrodes used for recording intracranial brain activity, such as ECoG electrodes placed on the neocortex, or intracortical electrodes for sensing sEEG, may be coupled to the external electrostimulator to provide multisite DBS at respective neural targets proximal to those intracranial electrodes. As will be discussed further below, such cortical stimulation may be used to modulate the ERs to DBS delivered via implantable lead electrodes (such as those shown in FIGS. 9A-9B) at respective neural targets (e.g., STN and GPi), and produce a desired therapeutic outcome in the patient.

Referring back to FIG. 8, the controller circuit 820 may include circuit sets comprising one or more other circuits or sub-circuits, such as a signal analyzer circuit 822 and a multisite stimulation controller 826. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 820 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

The signal analyzer circuit 822 may include an ER feature extraction circuit 823 that can extract one or more ER features from the ER sensed by the sensing circuit 810. Examples of the ER features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length can be measured as accumulated signal value differences of the sensed evoked response (or an epoch-averaged evoked response) over consecutive unit times (e.g., consecutive data sampling intervals) within the time window. The signal power can be measured as an area under the curve (AUC) of the sensed evoked response (or the epoch-averaged evoked response) within the time window.

In some examples, the signal analyzer circuit 822 may generate a spatial distribution of extracted signal features across the sensing locations of the sensing electrodes. For example, multiple sensing electrodes at or proximal to the first brain target may each sense respective ERs, and a spatial distribution of the ER features across the multiple sensing electrodes can be determined. In some examples, the spatial distribution may be determined by fitting the ER data to an ER distribution model, such as a Gaussian distribution model, a periodic or wrapped Gaussian distribution model, an exponential distribution model, a Poisson distribution model, a Weibull distribution, a regression model, or a non-parametric model such as a decision tree, a K-nearest neighbor model, a support vector machine (with Gaussian kernels for example), or artificial neural network, among others. The ER feature extraction circuit 823 may extract one or more features or parameters from the ER distribution model, including, for example, a mean value or a standard deviation of the ER features. In another example, the model features or parameters may include a morphological or statistical feature of the distribution model, such as an amplitude, a spatial location, or a width of a peak of the fitted model within a range defined by the plurality of sensing locations. In another example, the model features or parameters may include one or more of a positive peak amplitude (or a local maximum) or a negative peak amplitude (or a local minimum) of the fitted model within a range defined by the plurality of sensing locations. In yet another example, the model features or parameters may include a composite feature, such as a ratio of a positive peak amplitude to a negative peak amplitude of the fitted model within a range defined by the plurality of sensing locations.

In some examples, the sensing circuit 810 may sense an ER to DBS at a first brain target (DBS₁) in accordance with a first stimulation setting, and the ER feature extraction circuit 823 may extract features from the ER to DBS₁ (hereinafter referred to as “ER₁ features”). The ER₁ features may be used to guide DBS at a second brain target (DBS₂), as will be discussed below. The signal analyzer circuit 822 may compare the ER₁ features (or the distribution of ER₁ features, or ER₁ distribution model features or parameters) to one or more acceptance criteria 832 to determine whether a match to a desired or target ER can be found. In some examples, the acceptance criteria may be set, modulated, inspected, accepted by the clinical user, including ahead of or during operation. In some examples, the signal analyzer circuit 822 may accumulate the sensed ERs obtained in multiple stimulation-ER recording sessions during which stimulation pulses are delivered via a particular stimulating electrode with varying stimulation parameter settings (e.g., stimulation amplitude, frequency, or pulse width), determine the ER distribution model (or the model features or parameters from the accumulated ERs, and compare the ER distribution model or the model features or parameters to the acceptance criteria 832. The acceptance criteria 832 can be provided by a user such as via the user interface 850. Alternatively, the acceptance criteria 832 can be predetermined and stored in a storage device 830 accessible by the signal analyzer circuit 822. In an example, the acceptance criteria 832 may include user-provided acceptance bounds (e.g., upper and lower bounds, location bounds, properties bounds such presence, absence, or value of a feature) of the model features or parameters. In another example, the acceptance criteria 832 may include a target ER distribution representing a patient-specific ER distribution or a population-based ER distribution. In an example, the target ER distribution may be selected to relieve symptoms or for other goals such as lead placement for disease-modifying therapy or co-therapy (e.g., leads that inject drugs or light), and side-effect avoidance. In some examples, the acceptance criteria may include one of a plurality of candidate ER templates indexed by region, clinical institution, group, participants information, implanter information, or symptom relief goals, and a target ER template can be selected from the plurality of candidate ER templates based at least in part on one or more of an identification of an institution where the patient is implanted or treated with the electrostimulation, a group, or the participants information, an identification of an implanter that implants the lead, or the sensed indication of symptom relief of the patient.

The multisite stimulation controller 826 may generate a control signal to the electrostimulator 840 to deliver multisite DBS based on the comparison of the ER₁ feature (or the distribution of ER₁ feature, or ER₁ distribution model feature or parameter) to the acceptance criteria 832. For example, if the signal analyzer circuit 822 determines that the ER₁ feature (or the distribution of ER₁ feature, or ER₁ distribution model feature or parameter) does not match the desired or target ER (such that the acceptance criteria are not met), then the multisite stimulation controller 826 may adjust the second stimulation setting for DBS at a second brain target (DBS₂). This may include, for example, guided placement of a second lead or selecting second one or more stimulation electrodes for stimulating the second brain target, and/or optimizing stimulation parameters at the second brain target. The first and the second brain targets may be in the same hemisphere of the brain. In an example, the first brain target is STN, and the second target is GPi in the same hemisphere of the brain. The DBS of the first and the second brain targets may be delivered via respective electrode or groups of electrodes in the same lead, or in separate leads, as described above with respect to FIGS. 9A-9B.

The DBS at the second brain target (DBS₂) may have an modulation effect on ER₁, resulting in a “compound” ER. The multisite stimulation controller 826 may adjust the stimulation setting of DBS₂ until the compound ER matches the desired or target ER, such that the acceptance criteria 832 have been met. In one example, the second stimulation setting of DBS₂ may be determined such that DBS₂ would boost ER₁ and therefore produce an enhanced compound ER. In another example, the stimulation setting of DBS₂ may be determined such that DBS₂ would reduce ER₁ and therefore produce a diminished compound ER.

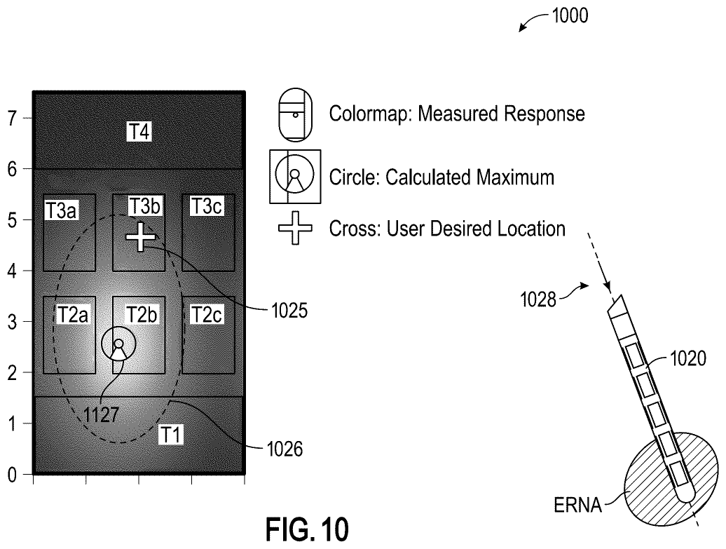

In some examples, the multisite stimulation controller 826 may determine or adjust the stimulation setting of DBS₂ (e.g., lead placement, stimulation parameters, and electrode selection) based on a therapeutic outcome. The therapeutic outcome may be evaluated by comparing a spatial distribution of ER to a desired ER spatial distribution. For example, the multisite stimulation controller 826 may adjust the stimulation setting of DBS₂ to reduce a discrepancy between a spatial distribution of the compound ER (i.e., ER₁ modulated by DBS of the second brain target) and the desired ER spatial distribution. In an example, the distribution of sensed ER can be depicted as a two dimensional (2D) hotspot view including a hotspot for the distribution of ERs. The hotspot represents an ER distribution center where the ER amplitude reaches a peak value. The multisite stimulation controller 826 may compare the measured hotspot based on the ER to DBS₁ to a user-defined target hotpot. The target hotspot represents desired location and amplitude of the peak of ER distribution, and corresponds to better therapeutic outcome. As will be described further below with respect to FIG. 10, the difference between the measured hotspot and the target hotpot may be used to optimize DBS₂, such as by adjusting the stimulation setting of DBS₂ until the measured hotspot of the compound ER matches the target hotspot (e.g., the difference in ER peak location and/or ER peak amplitude falls below a threshold value). This ER-guided multisite DBS may improve the therapeutic efficacy of multisite DBS.

The multisite stimulation controller 826 may be implemented as a proportional integral (PI) controller, a proportional-integral-derivative (PID) controller, or other suitable controller that takes the comparison of the sense ERs (or features or a distribution of the features thereof) to the acceptance criteria 852 as a feedback on the adjustment of stimulation settings. The types of data, and the recordings used to produce them, may vary regarding the type of acceptance criteria and operations employed. For example, ER data used to drive decisions about the electrode selection and configuration may differ from data and evoke/record configurations used to compare to acceptance criteria and use as control signal for amplitude adjustment. One ER measurement may be used to inform lead positioning (e.g., by sweeping a non-therapeutic sampling pulse across the space of the lead electrodes), another ER measurement may be used to determine or adjust a stimulation parameter (e.g., by sweeping a therapeutic sampling pulse across amplitudes).

The electrostimulator 840 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In some examples, the electrostimulator 840 may be configured to deliver multisite DBS at respective brain targets, such as STN and GPi, in accordance with respective stimulation settings. The multisite DBS may be delivered substantially simultaneously, sequentially, or in accordance with a predetermined pattern to respective brain targets.

The electrostimulator 840 can be an implantable module, such as incorporated within the IPG 10. Alternatively, the electrostimulator 840 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 840 can deliver stimulation in accordance with the adjusted therapy setting.

In some examples, the multisite stimulation controller 826 can generate a recommendation to the user to reposition the lead or to adjust the device setting (e.g., a programmable parameter of the electrostimulator 840). The repositioning of the lead or the adjustment of the device setting can cause the sensed ERs to align or more favorably compare to the acceptance criteria (e.g., an ER template) during an implantation procedure. In some examples, the multisite stimulation controller 826 may determine or modify therapeutic stimulation settings based on the sense ERs or features or a distribution of the features thereof. The electrostimulator 840 may deliver therapeutic stimulation (e.g., DBS) in accordance with the determined or modified therapeutic stimulation settings.

In some embodiments the display may provide a suggestion to the user to adjust stimulation parameters to cause the since developed responses to more favorably compare to the acceptance criteria (e.g., an ER template). The recommendation can be displayed on the user interface 850. The user interface 850 can be a portable (e.g., handheld) device, such as the RC 45 or a smartphone (with executable software application) operable by the patient at his or her home without requiring extra clinic visits or consultation with a device expert. In another example, the user interface 850 can be a programmer device, such as the CP 50. In addition to the recommendation for lead replacement, other information may be displayed on the user interface 850 including, by way of example and not limitation, one or more of the sensed ERs (including, for example, before and/or after filtering), ER features, distribution of ER features, the acceptance criteria (e.g., one or more ER templates), or the comparison between the sensed ERs and the acceptance criteria.

In some examples, the user interface 850 allows a physician to remotely review therapy settings and treatment history, consult with the patient to obtain information including side effects or symptoms associated with or produced by the electrostimulation, perform remote programming of the electrostimulator 840, or provide other treatment options to the patient. The user interface 850 can allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program can include a set of stimulation parameters with respective pre-determined values. In some examples, the user interface 850 can include a display to display textually or graphically information provided by the user via an input unit, and device settings including, for example, feature selection, sensing configurations, signal pre-processing settings, therapy settings, optionally with any intermediate calculations. In an example, the user interface 850 may present to the user an “optimal” or improved therapy setting, such as determined based on a closed-loop or adaptive feedback control of electrostimulation based on a selected evoked response signal feature, in accordance with various embodiments discussed in this document. In some examples, the user can use the user interface 850 to provide feedback on a neuromodulation therapy, including, for example, side effects or symptoms arise or persist associated with the neurostimulation, or severity of the symptom or a side effect.

FIG. 10 illustrates, by way of example and not limitation, a display 1000 on a user interface showing a comparison between a user-defined ER target location 1025 and a calculated ER distribution center 1027, overlaid upon a depiction of a portion of electrodes on a lead 1020, such as ring electrodes T1 and T4, segmented electrodes T2a, T2b, T2c, and segmented electrodes T3a, T3b, T3c, as shown in FIG. 10. The user-defined ER target location 1025 is an example of the acceptance criteria 832, and can represent a target ER distribution center. The calculated ER distribution center 1027 can be determined from a distribution of the ERs sensed by the signal analyzer circuit 822. The distribution of the sensed ERs can be estimated along the longitudinal direction and about the rotational direction. The distribuion of the sensed ERs can be depicted as a two dimensional (2D) hotspot view 1026 that provides an indicator (e.g., a heatmap shown as a colormap or a grayscale map) for a hotspot for the distribution of ERs. The calculated ER distribution center 1027 can represent an ER distribution peak amplitude at the peak location, such as a peak of a Gaussian distribution. In an example, the calculated ER distribution center 1027 can be determined from a distribution of the ER to DBS of the first brain target (DBS₁). Based on the comparison of the user-defined ER target location 1025 and the calculated ER distribution center 1027, a recommendation 1028 may be provided to the user to adjust a stimulation setting of DBS at a second brain target (DBS₂) different than the first brain target. The adjustment of stimulation setting of DBS₂ may include adjustment of lead placement or stimulation parameters, such that DBS₂ delivered in accordance with the adjusted stimulation setting would modulate the ER to DBS₁ (i.e., ER₁) and cause the calculated ER distribution center 1027 to more closely correspond to the user-defined ER target location 1025 (e.g., the calculated ER distribution center 1027 falls within a predetermined proximity to the user-defined ER target location 1025). The user-defined ER target location 1025 may be presented on a representation of lead electrodes. In some examples, the user interface 850 can determine a distance between the calculated ER distribution center 1027 and the user-defined ER target location 1025, and provide said distance to the user. During lead implantation, the calculated ER distribution center 1027 can be updated substantially in real time with adjustment of stimulation setting of DBS₂. A comparison between the user-defined ER target location 1025 and the updated calculated ER distribution center 1027, including the distance therebetween, can be displayed to the user to guide lead implantation.

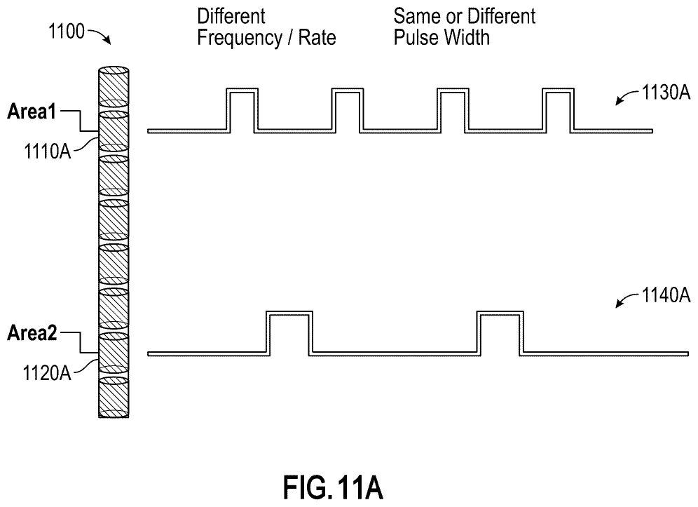

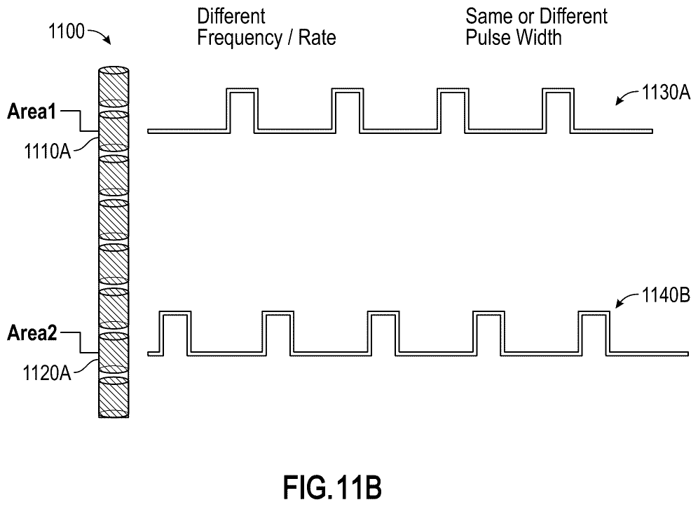

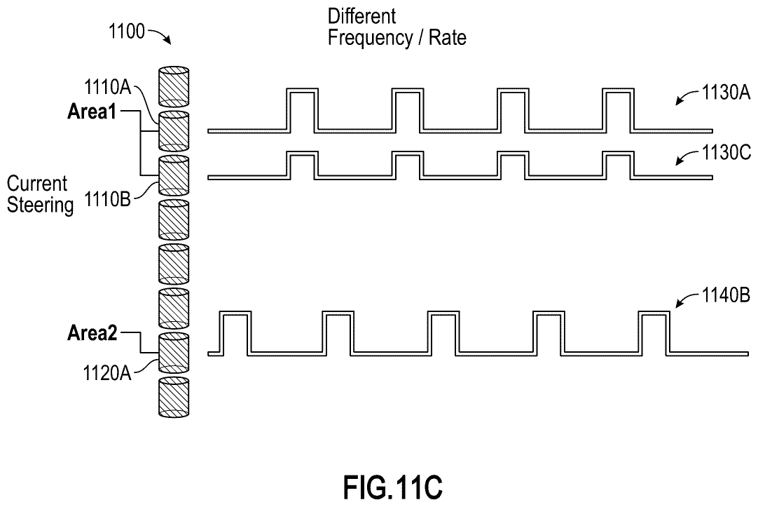

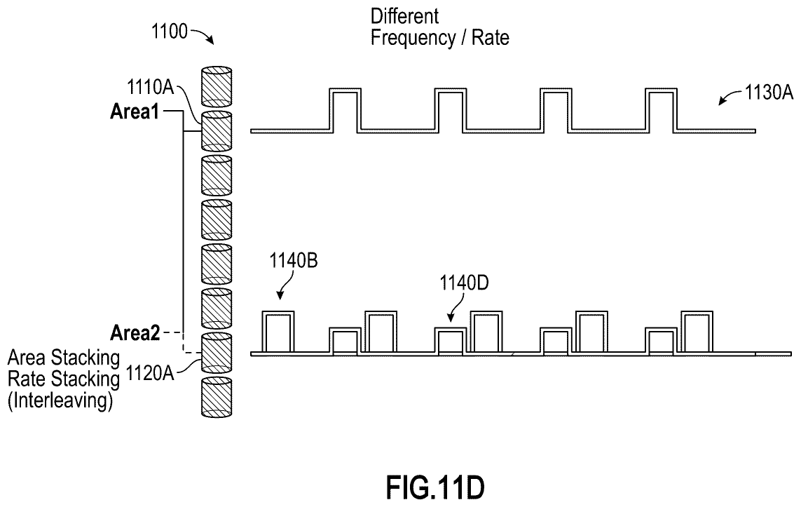

FIGS. 11A-11D illustrate, by way of example and not limitation, various stimulation patterns that may be used in mutlisite DBS, such as delivered via respective electrodes or groups of electrodes on the same single lead or distributed between two or more leads, as described above with respect to FIGS. 9A-9B. In particular, FIG. 11A illustrates DBS of a first target (“DBS₁” 1130A) via a first electrode 1110A on a lead 1100, and DBS of a second target (“DBS₂” 1140A) via a second electrode 1120A on the same lead 1100. In this example, DBS₁ 1130A and DBS₂ 1140A have different pulse rate (also referred to as pulse frequency, i.e., number of pulses per unit time), but may have the same or different pulse width. FIG. 11B similarly shows a stimulation pattern including DBS₁ 1130A of the first target via the first electrode 1110A, and DBS₂ 1140B of the second target via the second electrode 1120A on the same lead 1100. DBS₁ 1130A and DBS₂ 1140B may have the same or different pulse width. In contrast to the stimulation pattern shown in FIG. 11A, here DBS₁ 1130A and DBS₂ 1140B have the same pulse rate. FIG. 11C illustrates a modification of the simulation pattern shown in FIG. 11B, where DBS is delivered to two electrodes 1110A and 1110B both at or proximal to the first target, as opposed to only one electrode 1110A as shown in FIG. 11B. In this example, DBS at electrodes 1110A and 1110B are delivered in a current steering mode, where stimulation current is delivered to two (or more) electrodes at substantially the same time. In the illustrated example, DBS 1130A at electrode 1110A and DBS 1130C at electrode 1110B have the same pulse rate and timing, but different stimulation amplitudes. Similar to FIG. 11B, DBS₂ 1140B at the second target is delivered via the second electrode 1120A. FIG. 11D illustrates another modification of the multisite DBS simulation pattern shown in FIG. 11B. In this example, DBS₁ 1130A is delivered to the first target via electrode 1110A. DBS₂ of the second target, delivered via second electrode 1120A, comprises two DBS pulse trains: DBS 1140B as similarly shown in FIG. 11B, and another DBS 1140D with the same pulse rate as DBS₁ 1130A but different pulse amplitude and timing. The stimulation pattern at the second target as such is referred to as an interleaving mode, because DBS 1140B and DBS 1140D are delivered to the same electrode (e.g., electrode 1120A) but at different times, and one pulse train is interleved with another pulse train. In some examples, three or more DBS pulse trains with the same pulse rates may be applied to the same electrode electrode (e.g., electrode 1120A) in an interleaving mode.

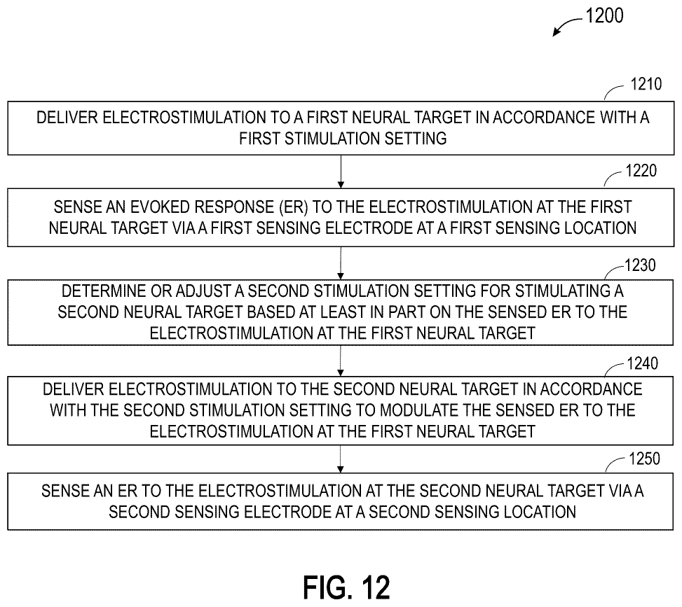

FIG. 12 illustrates, by way of example and not limitation, a method 1200 of providing evoked response (ER)-guided multisite DBS at various brain targets. The method 1200 may be carried out using a medical system such as the neuromodulation system 800. In an example, the method 1200 may be implemented in a programmer device such as RC 45 or CP 50 in communication with an electrostimulator such as IPG 10 or electrostimulator 840. In some examples, the method 1200 may alternatively be used to provide ER-based multisite stimulation at other neural targets, such as spinal cord stimulation (SCS) at multiple spinal neural targets.

At 1210, electrostimulation may be delivered to a first neural target in accordance with a first stimulation setting. In an example of DBS, the first neural target may be a brain target such as STN or GPi. Electrostimulation such as DBS may be delivered via stimulating electrodes selected from electrodes associated with at least one lead, such as the non-directional lead 301A or the directional lead 301B.

At 1220, an evoked response (ER) to the electrostimulation at the first neural target may be sensed via a first sensing electrode positioned at a first sensing location. The first sensing electrode may be selected from electrodes associated with the same lead used for providing electrostimulation, or alternatively, from electrodes associated with different leads, as described above with respect to FIGS. 9A-9B. The first sensing electrode for sensing the ER to DBS at the first neural target may be positioned at a location distant away from the first neural target being stimulated. In an example, the first neural target being stimulated is an STN target, and the ER to STN DBS may be sensed at a location at or proximal to GPi. In another example, the first neural target being stimulated is a GPi target, and the ER to GPi DBS may be sensed at a location at or proximal to STN in the same brain hemisphere.

In some examples, at least some sensing electrodes or the stimulating electrodes may be selected from electrodes other than the electrodes on an implantable lead, such as skin patch electrodes. In some examples, intracranial brain activity, such as intracranial electroencephalograph (iEEG) may be recorded via electrode placed directly on the neocortex to record electrocorticography (ECoG), or placed intracortically to record stereoelectroencephalography (sEEG).

At 1630, a second stimulation setting for stimulating a second neural target different from the first neural target may be determined or adjusted based at least in part on the sensed ER to the electrostimulation at the first neural target. In the context of multisite DBS, the first and the second neural targets may be targets in the same hemisphere of the brain. In an example, the first brain target is STN, and the second target is GPi in the same brain hemisphere.

The ER-guided determination or adjustment of the second stimulation setting may involve extracting features from the sensed ER to DBS at a first brain target (hereinafter referred to as “ER₁ features”). Examples of the ER features may include a signal amplitude, magnitude, peak value, value range, a signal curve length, or a signal power or RMS value of an ER signal within a time window, such as the epoch-averaged ERs. In some examples, a spatial distribution of extracted signal features across the sensing locations of the sensing electrodes may be determined, and one or more features or parameters may be extracted from the ER distribution model, including, for example, a mean value or a standard deviation of the ER features. The ER₁ features (or the distribution of ER₁ features, or ER₁ distribution model features or parameters) may be compared to one or more acceptance criteria to determine whether a match to a desired or target ER can be found. The acceptance criteria can be provided by a user, or predetermined and stored in a storage device. In an example, the acceptance criteria may include user-provided acceptance bounds (e.g., upper and lower bounds, location bounds, properties bounds such presence, absence, or value of a feature) of the model features or parameters. In another example, the acceptance criteria may include a target ER distribution representing a patient-specific ER distribution or a population-based ER distribution. In an example, the target ER distribution may be selected to relieve symptoms or for other goals such as lead placement for disease-modifying therapy or co-therapy (e.g., leads that inject drugs or light), and side-effect avoidance.

At 1640, electrostimulation may be delivered to the second neural target in accordance with the second stimulation setting to modulate the sensed ER to the electrostimulation at the first neural target, and to produce a desired therapeutic outcome in the patient. For example, if the features extracted from ER₁ (or from the distribution of ER₁ features, or ER₁ distribution model features or parameters) do not match the desired or target ER (and thus the acceptance criteria are not met), then the second stimulation setting (for stimulating the second brain target) may be adjusted, which may include guided placement of a second lead or selecting second one or more stimulation electrodes for stimulating the second brain target, and/or optimizing stimulation parameters at the second brain target.

At 1650, an ER to the electrostimulation at the second neural target may be sensed via a second sensing electrode positioned at a second sensing location. In multisite DBS, stimulation at the second brain target may have an modulation effect on ER₁, resulting in a “compound” ER. The second stimulation setting (for stimulating the second brain target) may be adjusted until the compound ER matches the desired or target ER, such that the acceptance criteria have been met. In one example, the second stimulation setting may be determined such that the DBS of the second brain target would boost ER₁, and therefore produce an enhanced compound ER. In another example, the second stimulation setting may be determined such that the DBS of the second brain target would reduce ER₁, and therefore produce a diminished compound ER. In some examples, the second stimulation setting (e.g., lead placement, stimulation parameters, and electrode selection) may be adjusted based on a therapeutic outcome. The therapeutic outcome may be evaluated by comparing a spatial distribution of ER to a desired ER spatial distribution. For example, the second stimulation setting may be adjusted to reduce a discrepancy between a spatial distribution of the compound ER (i.e., ER₁ modulated by DBS of the second brain target) and the desired ER spatial distribution. In an example, the distribuion of sensed ER can be depicted as a two dimensional (2D) hotspot view including a hotspot for the distribution of ERs. The measured hotspot based on ER₁ may be compared to a user-defined target hotpot. The target hotspot represents desired location and amplitude of the peak of ER distribution, and corresponds to better therapeutic outcome. In some examples, the difference between the measured hotspot and the target hotpot may be used to optimize the second stimulation setting (for stimulating the second brain target), such as by adjusting one or more stimulation parameters or selecting different stimulating electrodes) until the measured hotspot of the compound ER matches the target hotspot (e.g., the difference in ER peak location and/or ER peak amplitude falls below a threshold value). The ER-guided multisite DBS as described may improve therapeutic efficacy of DBS.

In some examples, the result of the comparison of the spatial distribution of ER and the desired ER spatial distribution (such as the comparison between the measured hotspot based on ER₁ and the user-defined target hotpot) may be presented to a user (e.g., a clinician), such as displayed on a user interface. Based on such comparison, a recommendation can be provided to the user, such as a recommendation to reposition the at least one lead, such as pushing, pulling, shifting, or rotating the lead to achieve a desired target response, or a recommendation to adjust the stimulation setting. During the repositioning of the at least one lead and/or the adjustment of stimulating setting, evoked responses may be sensed, and ER features and/or distributions may be determined, and comparison to the acceptance criteria can be updated in substantially real time and displayed to the user. The user may continuate repositioning the at least one lead and/or adjusting the stimulating setting until the sensed ER compares more favorably to the acceptance criteria. The sense ER or features or a distribution of the ER feature generated therefrom may be used as feedback to further modify therapeutic stimulation settings.

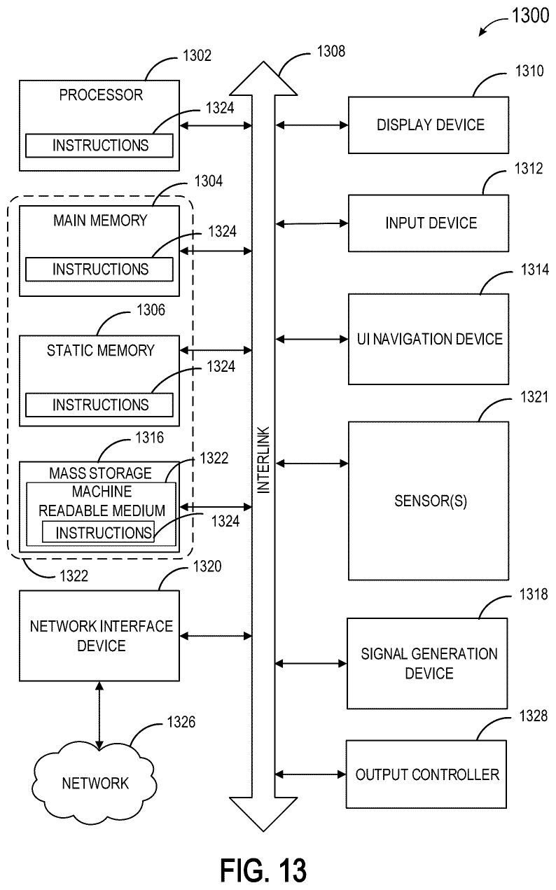

FIG. 13 illustrates generally a block diagram of an example machine 1300 upon which any one or more of the techniques (e.g., methodologies) discussed herein may perform. Portions of this description may apply to the computing framework of various portions of the neuromodulation device or the external programmer device.

In alternative examples, the machine 1300 may operate as a standalone device or may be connected (e.g., networked) to other machines. In a networked deployment, the machine 1300 may operate in the capacity of a server machine, a client machine, or both in server-client network environments. In an example, the machine 1300 may act as a peer machine in peer-to-peer (P2P) (or other distributed) network environment. The machine 1300 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1300 may include a hardware processor 1302 (e.g., a central processing unit (CPU), a graphics processing unit (GPU), a hardware processor core, algorithm specific ASIC, or any combination thereof), a main memory 1304 and a static memory 1306, some or all of which may communicate with each other via an interlink (e.g., bus) 1308. The machine 1300 may further include a display unit 1310 (e.g., a raster display, vector display, holographic display, etc.), an alphanumeric input device 1312 (e.g., a keyboard), and a user interface (UI) navigation device 1314 (e.g., a mouse). In an example, the display unit 1310, input device 1312 and UI navigation device 1314 may be a touch screen display. The machine 1300 may additionally include a storage device (e.g., drive unit) 1316, a signal generation device 1318 (e.g., a speaker), a network interface device 1320, and one or more sensors 1321, such as a global positioning system (GPS) sensor, compass, accelerometer, or other sensors. The machine 1300 may include an output controller 1328, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1316 may include a machine-readable medium 1322 on which is stored one or more sets of data structures or instructions 1324 (e.g., software) embodying or utilized by any one or more of the techniques or functions described herein. The instructions 1324 may also reside, completely or at least partially, within the main memory 1304, within static memory 1306, or within the hardware processor 1302 during execution thereof by the machine 1300. In an example, one or any combination of the hardware processor 1302, the main memory 1304, the static memory 1306, or the storage device 1316 may constitute machine readable media.

While the machine-readable medium 1322 is illustrated as a single medium, the term “machine readable medium” may include a single medium or multiple media (e.g., a centralized or distributed database, and/or associated caches and servers) configured to store the one or more instructions 1324.

The term “machine readable medium” may include any medium that is capable of storing, encoding, or carrying instructions for execution by the machine 1300 and that cause the machine 1300 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1324 may further be transmitted or received over a communication network 1326 using a transmission medium via the network interface device 1320 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1320 may include one or more physical jacks (e.g., Ethernet, coaxial, or phone jacks) or one or more antennas to connect to the communication network 1326. In an example, the network interface device 1320 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1300, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A neuromodulation system, comprising:
at least one lead including a plurality of electrodes;
an electrostimulator configured to provide electrostimulation to neural targets of a patient via one or more of the plurality of electrodes of the at least one lead;
a sensing circuit configured to sense an evoked response (ER) to the electrostimulation; and
a controller circuit operably connected to the electrostimulator and the sensing circuit, the controller circuit configured to:
in response to the electrostimulation delivered to a first neural target in accordance with a first stimulation setting, collect an ER sensed via the sensing circuit from a first sensing electrode positioned at a first sensing location;
based at least in part on the sensed ER to the electrostimulation at the first neural target, determine or adjust a second stimulation setting for stimulating a second neural target of the patient different from the first neural target;
generate a control signal to the electrostimulator to provide electrostimulation at the second neural target in accordance with the second stimulation setting to modulate the sensed ER to the electrostimulation at the first neural target, and to produce a desired therapeutic outcome in the patient; and
collect an ER to the electrostimulation at the second neural target sensed via the sensing circuit from a second sensing electrode positioned at a second sensing location.

2. The neuromodulation system of claim 1, wherein the controller circuit is configured to control the electrostimulator to deliver respective electrostimulations to the first and the second neural targets substantially simultaneously, or sequentially with a specific time offset in accordance with respective the first and the second stimulating settings.

3. The neuromodulation system of claim 1, wherein the first neural target of electrostimulation is a subthalamic nucleus (STN) target in a hemisphere of a brain, and the second neural target of electrostimulation is a globus pallidus internus (GPi) target in the same hemisphere of the brain.

4. The neuromodulation system of claim 3, wherein:
the first sensing electrode is positioned at a GPi sensing location to sense the ER to electrostimulation of the STN target; and
the second sensing electrode is positioned at an STN sensing location to sense the ER to electrostimulation of the GPi target.

5. The neuromodulation system of claim 1, further comprising an external electrostimulator operatively coupled to one or more cortical electrodes or one or more stereotactic electrodes to provide cortical stimulation to modulate one or more of the sensed ER to the electrostimulation at the first neural target or the sensed ER to the electrostimulation at the second neural target, and to produce the desired therapeutic outcome in the patient.

6. The neuromodulation system of claim 1, wherein the electrostimulator is electrically coupled to a multielectrode lead, the electrostimulator configured to provide electrostimulation to the first neural target via a first electrode on the multielectrode lead, and to provide electrostimulation to the second neural target via a second electrode on the same multielectrode lead.

7. The neuromodulation system of claim 1, wherein the at least one lead includes distinct first and second leads each including one or more electrodes,
wherein the electrostimulator is configured to provide electrostimulation to the first neural target via a first electrode on the first lead, and to provide electrostimulation to the second neural target via a second electrode on the second lead.

8. The neuromodulation system of claim 1, wherein at least one of the sensed ER to the electrostimulation at the first neural target or the sensed ER to the electrostimulation at the second neural target includes an electrocorticography (ECoG) or a stereoelectroencephalography (sEEG) sensed via one or more cortical electrodes or one or more stereotactic electrodes.

9. The neuromodulation system of claim 1, wherein the controller circuit is configured to determine or adjust the second stimulation setting such that the electrostimulation at the second neural target in accordance with the second stimulation setting reduces the ER to the electrostimulation at the first neural target.

10. The neuromodulation system of claim 1, wherein the controller circuit is configured to determine or adjust the second stimulation setting such that the electrostimulation at the second neural target in accordance with the second stimulation setting enhances the ER to the electrostimulation at the first neural target.

11. The neuromodulation system of claim 1, wherein to determine or adjust the second stimulation setting based at least in part on the sensed ER to the electrostimulation at the first neural target, the controller circuit is configured to:
evaluate a therapeutic outcome using the sensed ER to the electrostimulation at the first neural target; and
based on the evaluation of the therapeutic outcome, determine or adjust the second stimulation setting including one or more stimulation parameters or one or more electrodes selected from the plurality of electrodes of the at least one lead for electrostimulation.

12. The neuromodulation system of claim 11, wherein to evaluate the therapeutic outcome includes to compare a spatial distribution of the sensed ER to the electrostimulation at the first neural target to a desired ER spatial distribution,
wherein the controller circuit is configured to determine or adjust the second stimulation setting to reduce a discrepancy between the spatial distribution of the sensed ER to the electrostimulation at the second neural target and the desired ER spatial distribution.

13. A method of providing neurostimulation to neural targets of a patient via a neuromodulation system that comprises an electrostimulator and at least one lead coupled thereto, the method comprising:
delivering electrostimulation to a first neural target in accordance with a first stimulation setting using the electrostimulator;
sensing an evoked responses (ER) to the electrostimulation at the first neural target via a first sensing electrode positioned at a first sensing location;
via a controller circuit, determining or adjusting a second stimulation setting for stimulating a second neural target of the patient different from the first neural target based at least in part on the sensed ER to the electrostimulation at the first neural target;
delivering electrostimulation to the second neural target in accordance with the second stimulation setting to modulate the sensed ER to the electrostimulation at the first neural target, and to produce a desired therapeutic outcome in the patient; and
sensing an ER to the electrostimulation at the second neural target via a second sensing electrode positioned at a second sensing location.

14. The method of claim 13, wherein the electrostimulation of the first neural target and the electrostimulation of the second neural target are delivered substantially simultaneously, or sequentially with a specific time offset, in accordance with respective the first and the second stimulating settings.

15. The method of claim 13, wherein the first neural target of electrostimulation is a subthalamic nucleus (STN) target in a hemisphere of a brain, and the second neural target of electrostimulation is a globus pallidus internus (GPi) target in the hemisphere of the brain.

16. The method of claim 13, further comprising providing cortical stimulation via an external electrostimulator operatively coupled to one or more cortical electrodes or one or more stereotactic electrodes, the cortical stimulation modulating one or more of the sensed ER to the electrostimulation at the first neural target or the sensed ER to the electrostimulation at the second neural target, and producing the desired therapeutic outcome in the patient.

17. The method of claim 13, wherein the at least one lead includes distinct first and second leads each including one or more electrodes,
wherein the electrostimulation to the first neural target is delivered via a first electrode on the first lead,
wherein the electrostimulation to the second neural target is delivered via a second electrode on the second lead.

18. The method of claim 13, wherein at least one of the sensed ER to the electrostimulation at the first neural target or the sensed ER to the electrostimulation at the second neural target includes an electrocorticography (ECoG) or a stereoelectroencephalography (sEEG) sensed via one or more cortical electrodes or one or more stereotactic electrodes.

19. The method of claim 13, wherein the second stimulation setting is determined or adjusted such that the electrostimulation at the second neural target in accordance with the second stimulation setting produces a desired modulation of the ER to the electrostimulation at the first neural target, the desired modulation including reducing the ER to the electrostimulation at the first neural target, or enhancing the ER to the electrostimulation at the first neural target.

20. The method of claim 13, comprising comparing a spatial distribution of the sensed ER to the electrostimulation at the first neural target to a desired ER spatial distribution,
wherein the second stimulation setting is determined or adjusted to reduce a discrepancy between the spatial distribution of the sensed ER to the electrostimulation at the second neural target and the desired ER spatial distribution.
